(19) United States
(12) Patent Application Publication
(10) Pub. No.: US 2025/0099368 A1
(43) Pub. Date: Mar. 27, 2025

(54) CAPSULE MEDICATION DEVICE AND CAPSULE MEDICATION DEVICE CHARGING SYSTEM

(71) Applicant: Murata Manufacturing Co., Ltd., Nagaokakyo-shi (JP)

(72) Inventors: Takanori NAKAMURA, Nagaokakyo-shi (JP); Takahiro NAGAI, Nagaokakyo-shi (JP)

(21) Appl. No.: 18/976,532

(22) Filed: Dec. 11, 2024

Related U.S. Application Data
(63) Continuation of application No. PCT/JP2023/009768, filed on Mar. 14, 2023.

(30) Foreign Application Priority Data
Jun. 17, 2022 (JP) 2022-097828

(51) Int. Cl. A61K 9/00; A61B 5/00; A61M 31/00; H02J 50/10

(52) U.S. Cl. CPC A61K 9/0009; A61M 31/00; H02J 50/10; A61B 5/4833; A61M 2205/0294; A61M 2205/8243

(57) ABSTRACT

A capsule medication device is provided that includes a housing having a capsule shape, a substrate disposed inside the housing, a power receiving coil on the substrate, and a secondary battery that can be charged with power received by the power receiving coil. A gravity center of the capsule medication device 10 not located at a center of a cut face when the capsule medication device is cut along a plane that passes through the gravity center and is orthogonal to the longitudinal direction of the capsule medication device, and is located to overlap the substrate in a view along a direction orthogonal to the substrate.

### CROSS REFERENCE TO RELATED APPLICATIONS

The present application is a continuation of International Application No. PCT/JP2023/009768, filed Mar. 14, 2023, which claims priority to Japanese Patent Application No. 2022-097828, filed Jun. 17, 2022, the entire contents of each of which are hereby incorporated by reference.

### TECHNICAL FIELD

The present disclosure relates to a capsule medication device, and a capsule medication device charging system.

### BACKGROUND

There is a need for a method of checking whether a patient has taken a prescribed medicine and a method of checking what biological reaction may occur by a patient taking a medicine. In this regard, development of a medication device that transmits a signal from inside a body to the outside of the body after being taken with a medicine is currently in progress.

Japanese Patent No. 5730822 (the “'822 Patent”) describes a system that detects that a medicine has been taken, after a medication device containing the medicine has been taken, by causing a current to flow in a drive circuit included in the medication device via a conductive liquid, such as gastric juice, to transmit a signal that is then received by an external receiving device.

Moreover, a gastric acid battery, which causes a current to flow in a drive circuit via a conductive liquid such as gastric juice, is suitable for a system that requires power only for a short time, but is not suitable for a system, for example, that retains a medication device in a body for a long time to acquire biological information and transmit the acquired biological information to outside of the body. For such a system, a configuration may be suitable in which a small secondary battery capable of supplying power at a required timing is provided in a medication device.

However, when a small secondary battery is provided inside a medication device, a capsule-shaped medication device in particular, it is difficult to efficiently charge such a secondary battery.

### SUMMARY OF THE INVENTION

In view of the foregoing, it is an object of the present disclosure to provide a capsule medication device that provides for efficient charging of a secondary battery inside the capsule medication device. Moreover, a capsule medication device charging system is also provided.

Thus, according to an exemplary aspect, a capsule medication device is provided that includes a housing having a capsule shape; a substrate disposed inside the housing; a power receiving coil on the substrate; and a secondary battery configured to be charged with power received by the power receiving coil. In this aspect, a gravity center of the capsule medication device is not located at a center of a cut face when the capsule medication device is cut along a plane that passes through the gravity center and is orthogonal to a longitudinal direction of the capsule medication device. Moreover, the gravity center overlaps the substrate in a direction orthogonal to the substrate.

Moreover, in another exemplary aspect, a capsule medication device charging system is provided that includes a storage sheet including a plurality of storages and a metal cover that covers openings of the plurality of storages; the capsule medication device described above and that is stored in one of the plurality of storages of the storage sheet; and a charger that includes a power transmission coil. In this aspect, the storage sheet stores the capsule medication device in the one of the plurality of storages being placed on the charger.

In a capsule medication device of an exemplary aspect of the present disclosure, when the capsule medication device is placed on a charger including a power transmission coil, a power receiving coil and the power transmission coil do not have an orthogonal positional relationship, so that wireless power supply can be efficiently performed and a secondary battery can be efficiently charged. In addition, when the capsule medication device is placed on the charger including the power transmission coil, the relative positional relationship between the power transmission coil and the power receiving coil is uniquely determined, and thus variation in wireless charging can be suppressed.

According to a capsule medication device charging system of the present disclosure, charging is performed with high charging efficiency by only placing a storage sheet in which the capsule medication device is stored in a storage on the charger including the power transmission coil.

### BRIEF DESCRIPTION OF DRAWINGS

FIG. 1(a) is a side view schematically illustrating a capsule medication device according to one exemplary embodiment.

FIG. 1(b) is a sectional view schematically illustrating a configuration in a section of the capsule medication device illustrated in FIG. 1(a) taken along line 1B-1B.

FIG. 1(c) is a sectional view schematically illustrating a configuration in a section of the capsule medication device illustrated in FIG. 1(a) taken along line 1C-1C.

FIG. 2 is a plan view schematically illustrating the shape of a power receiving coil provided on a substrate.

FIG. 3 is a view schematically illustrating external shapes of a first split housing and a second split housing forming a housing.

FIG. 4 is a side sectional view schematically illustrating a configuration of a capsule medication device charging system.

FIG. 5(a) is a side sectional view schematically illustrating an orientation of the capsule medication device according to one exemplary embodiment, in which orientation the capsule medication device is charged.

FIG. 5(b) is a side sectional view schematically illustrating an orientation of the capsule medication device in which the center of a cut face on a plane that passes through the gravity center of the capsule medication device and is orthogonal to the longitudinal direction of the capsule medication device is located at the gravity center of the capsule medication device, in which orientation the capsule medication device is charged.

FIG. 5(c) is a side sectional view schematically illustrating an orientation of the capsule medication device in which the gravity center is not located at the center of the cut face and is not located to overlap the substrate in a view along a direction orthogonal to the substrate, in which orientation the capsule medication device is charged.

FIG. 6 is a view for explaining an angle θ between a line connecting the gravity center of the capsule medication device and the center of the cut face on the plane that passes through the gravity center of the capsule medication device and is orthogonal to the longitudinal direction of the capsule medication device, and a reference line that passes through the center of the cut face and extends in a direction orthogonal to the substrate.

FIG. 7 is a view for explaining the relationship between the angle θ between the line connecting the gravity center of the capsule medication device and the center of the cut face on the plane that passes through the gravity center of the capsule medication device and is orthogonal to the longitudinal direction of the capsule medication device, and the reference line that passes through the center of the cut face and extends in the direction orthogonal to the substrate, and an output voltage of a rectifying bridge circuit disposed subsequent to the power receiving coil.

### DETAILED DESCRIPTION

Hereinafter, features of the exemplary aspects of the present disclosure will be specifically described with reference to an exemplary embodiment.

A capsule medication device 10 of one exemplary embodiment is taken with a medicine and, for example, can transmit biological information outside the body. The medicine to be taken with the capsule medication device 10 may be any type of medicine as would be appreciated to one skilled in the art.

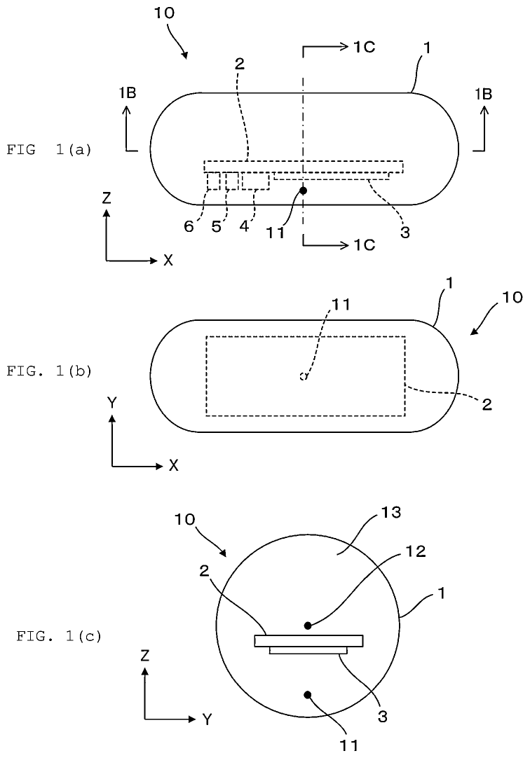

FIG. 1(a) is a side view schematically illustrating the capsule medication device 10 according to one exemplary embodiment. FIG. 1(b) is a sectional view schematically illustrating a configuration of the capsule medication device 10 illustrated in FIG. 1(a) taken along line 1B-1B. FIG. 1(c) is a sectional view schematically illustrating a configuration of the capsule medication device 10 illustrated in FIG. 1(a) taken along line 1C-1C. In FIGS. 1(a) to 1(c), X-axis direction is the longitudinal direction and Y-axis direction is the lateral direction of the capsule medication device 10. It is noted that Z axis is an axis orthogonal to the X axis and the Y axis.

The capsule medication device 10 of one exemplary embodiment includes a housing 1 having a capsule shape (e.g., an elongated shape in longitudinal direction), a substrate 2, a power receiving coil 3, and a secondary battery 4.

The housing 1 can be made of, for example, a biocompatible resin or a general-purpose resin having a surface coated with a biocompatible material. For example, an epoxy resin can be used as a biocompatible resin. A material preferably used for the housing 1 is a material that is discharged to the outside of the body without being dissolved by gastric acid or the like after the capsule medication device 10 is taken into the body.

As shown, the substrate 2 is disposed inside the housing 1. Moreover, one or a plurality of substrates 2 may be disposed inside the housing 1. For example, a substrate on which the power receiving coil 3 is provided, a substrate on which the secondary battery 4 is provided, and a substrate on which a control IC is provided may be collectively provided as the substrate 2.

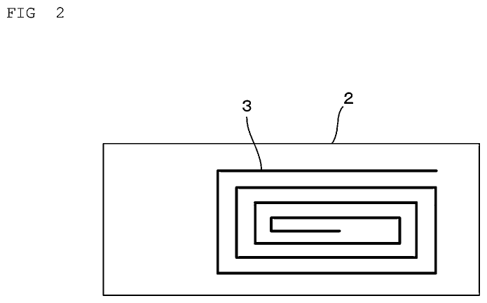

The power receiving coil 3 is configured to perform wireless power supply in pair with the power transmission coil and is provided on the substrate 2. The power receiving coil 3 can be made of, for example, copper. It is noted that wireless power supply may be performed using a technique using electromagnetic induction or magnetic field resonance. Magnetic field resonance causes a current to flow in the power receiving coil 3 by an effect of varying of the magnetic field generated by the power transmission coil and is thus a type of electromagnetic induction. FIG. 2 is a plan view schematically illustrating the shape of the power receiving coil 3 provided on the substrate 2. Although not illustrated in FIG. 2, the power receiving coil 3 is connected to the secondary battery 4.

In the exemplary aspect, the secondary battery 4 can be charged with the power received by the power receiving coil 3. The secondary battery 4 can be any chargeable and dischargeable battery, and is, for example, an all-solid-state battery having a solid electrolyte. The secondary battery 4 is provided, for example, on the substrate 2 on which the power receiving coil 3 is provided. However, the secondary battery 4 is not necessarily provided on the substrate 2 where power receiving coil 3 is provided in an alternative aspect.

The capsule medication device 10 may further include a biological information acquisition unit 5 and a signal transmission unit 6. Power is supplied from the secondary battery 4 to the biological information acquisition unit 5 and the signal transmission unit 6. FIG. 1 illustrates a configuration in which the biological information acquisition unit 5 and the signal transmission unit 6 are provided on the substrate 2 where the power receiving coil 3 is provided. However, the biological information acquisition unit 5 and the signal transmission unit 6 are not necessarily provided on the substrate 2 in an alternative aspect.

In an exemplary aspect, the biological information acquisition unit 5 is configured to acquire biological information, such as the location inside the body of the capsule medication device 10 taken with a medicine and the temperature in the body. For example, the biological information acquisition unit 5 includes a timing unit configured to measure time such that an elapsed time can be measured after the capsule medication device 10 is taken into the body, and further configured to estimate the location of the capsule medication device 10 in the body according to the measured time. In another example, the biological information acquisition unit 5 includes a temperature sensor configured to detect the temperature in the body. It is noted that the biological information may include any piece of information related to a living body, and the biological information acquisition unit 5 can be configured to acquire any piece of biological information.

The signal transmission unit 6 is further configured to transmit the biological information acquired by the biological information acquisition unit 5. That is, after the capsule medication device 10 is taken into the body, the signal transmission unit 6 transmits the biological information acquired by the biological information acquisition unit 5 outside the body. The signal transmission unit 6 is, for example, a piezoelectric element, and is configured to transmit biological information as an ultrasonic signal. Material that can be used for the piezoelectric element include lead zirconate titanate (PZT), barium titanate (BT), and potassium sodium niobate (KNN). In such a case, the capsule medication device 10 includes an IC for controlling the piezoelectric element. When the signal transmission unit 6 is a piezoelectric element, biological information can be reliably transmitted to the outside of the body as an ultrasonic signal. The signal transmitted from the signal transmission unit 6 is received by a signal receiving unit provided outside the body.

When the signal transmission unit 6 is a piezoelectric element and a vibration mode is a lateral displacement type (e.g., a d31 mode) for transmitting an ultrasonic signal, a longer piezoelectric element can generate a stronger ultrasonic signal. The capsule medication device 10 of the present exemplary embodiment having a capsule shape can accommodate a long piezoelectric element and transmit a strong ultrasonic signal.

However, the signal transmission unit 6 is not limited to a piezoelectric element. For example, the signal transmission unit 6 may be configured to transmit biological information by wireless communication.

As described above, power is supplied from the secondary battery 4 to the biological information acquisition unit 5 and the signal transmission unit 6. By supplying power from the secondary battery 4 instead of from a gastric acid battery to the biological information acquisition unit 5, the biological information acquisition unit 5 can be configured to acquire biological information at any location in the body. By supplying power from the secondary battery 4 instead of from a gastric acid battery to the signal transmission unit 6, the signal transmission unit 6 can be configured to transmit biological information at any location in the body.

FIG. 1(c) illustrates a cut face 13 (e.g., a planar cross-sectional cut or a cross-sectional plane) on a plane that passes through a gravity center 11 of the capsule medication device 10 and is orthogonal to the longitudinal direction of the capsule medication device 10. In the present exemplary embodiment, the cut face 13 is circular as illustrated in FIG. 1(ck). The gravity center 11 of the capsule medication device 10 can be obtained by a known method.

In the capsule medication device 10 according to the present exemplary embodiment, the gravity center 11 of the capsule medication device 10 is not located at a center 12 of the cut face 13 on a plane that passes through the gravity center 11 and is orthogonal to the longitudinal direction of the capsule medication device 10 as illustrated in FIG. 1(c). Moreover, the gravity center 11 overlaps the substrate 2 in a view along a direction orthogonal to the substrate 2 as illustrated in FIG. 1(b).

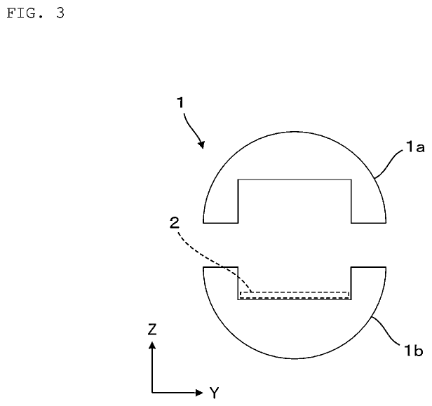

As an example, the housing 1 is configured by assembling a first split housing 1a and a second split housing 1b. FIG. 3 illustrates the first split housing 1a and the second split housing 1b before being assembled. In FIG. 3, as will be described later, the substrate 2 is indicated by a dotted line to show an example position of the substrate 2.

The first split housing 1a and the second split housing 1b have shapes formed by splitting the housing 1 by a plane that passes through the center of the housing 1 in the lateral direction thereof and is parallel to the longitudinal direction thereof. The first split housing 1a and the second split housing 1b can be manufactured by, for example, injection molding or a manufacturing method using a 3D printer. The first split housing 1a and the second split housing 1b can be assembled using an adhesive or the like as would be appreciated to one skilled in the art.

To locate the gravity center 11 of the capsule medication device 10 not on the center 12 of the cut face 13 described above, for example, the first split housing 1a and the second split housing 1b are formed to have different weights. For example, by forming the second split housing 1b to be heavier than the first split housing 1a, the gravity center 11 of the capsule medication device 10 can be adjusted to be within the second split housing 1b. As described above, the housing 1 is configured by assembling the first split housing 1a and the second split housing 1b, such that the first split housing 1a and the second split housing 1b are different in weight, so that the gravity center 11 of the capsule medication device 10 can be readily set to a desired location.

To form the first split housing 1a and the second split housing 1b to have different weights, for example, a weight may be provided in either one of the first split housing 1a and the second split housing 1b. To locate the gravity center 11 of the capsule medication device 10 to overlap the substrate 2 in a view along a direction orthogonal to the substrate 2, at least a portion of the weight is preferably located to overlap the substrate 2, more preferably, the whole weight is located to overlap the substrate 2, in a view along a direction orthogonal to the substrate 2.

As illustrated in FIG. 3, the substrate 2 may be provided inside the second split housing 1b or may be provided inside the first split housing 1a. Moreover, the substrate 2 may be disposed inside the second split housing 1b or the first split housing 1a by fitting, or may be disposed using a screw, an adhesive, or a double-sided tape.

Instead of providing a weight to any one of the first split housing 1a and the second split housing 1b, a weight may be provided on the substrate 2, and the substrate 2 provided with the weight may be disposed in either one of the split housings, that is the first split housing 1a and the second split housing 1b.

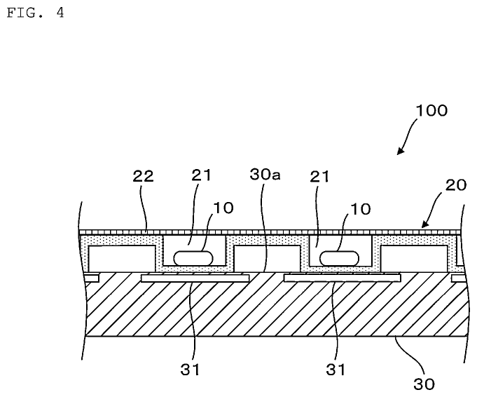

FIG. 4 is a side sectional view schematically illustrating a configuration of a capsule medication device charging system 100. The capsule medication device charging system 100 includes the capsule medication device 10, a storage sheet 20, and a charger 30.

The storage sheet 20 includes a plurality of storages 21 and a metal cover 22 that covers openings of a plurality of storages 21. The storage sheet 20 is, for example, a press through pack sheet (PTP sheet). Of the storage sheet 20, a portion forming the storage 21 is made of resin, for example, and the metal cover 22 is made of aluminum, for example.

The capsule medication device 10 is stored in the storage 21 of the storage sheet 20. FIG. 4 illustrates the capsule medication device 10 put through the opening of the storage 21 to be stored and the opening covered with the metal cover 22. As illustrated in FIG. 4, one capsule medication device 10 is stored in each storage 21. However, a plurality of the capsule medication devices 10 may be stored in one storage 21 as would be appreciated to one skilled in the art.

One storage 21 can be configured to store a medicine and the capsule medication device 10 to be taken at a time. If there is a plurality of medicines to be taken at a time, a plurality of medicines and one capsule medication device 10 may be stored in one storage 21 in this aspect.

The secondary battery 4 of the capsule medication device 10 stored in the storage 21 of the storage sheet 20 can be charged by the charger 30. The charger 30 has a power transmission coil 31 for wireless power supply. A plurality of power transmission coils 31 or only one power transmission coil 31 may be provided to the charger 30 in various exemplary aspects. When the secondary battery 4 of the capsule medication device 10 is charged, the storage sheet 20 storing the capsule medication device 10 in the storage 21 is placed on a placement surface 30a of the charger 30. As illustrated in FIG. 4, a portion of the storage sheet 20 that is brought into contact with the charger 30 is in the opposite side of the metal cover 22. In this state, when a current flows in the power transmission coil 31 of the charger 30, electromagnetic induction causes a current to flow in the power receiving coil 3 of the capsule medication device 10, whereby the secondary battery 4 is charged.

The power transmission coil 31 provided to the charger 30 is preferably at the placement surface 30a on which the storage sheet 20 is placed or at a location close to the placement surface 30a. Providing the power transmission coil 31 at the placement surface 30a or at a location close to the placement surface 30a of the charger 30 shortens the distance between the power transmission coil 31 and the power receiving coil 3 of the capsule medication device 10, which thereby performs wireless charging with high charging efficiency.

As described above, the portion of the storage sheet 20 that is brought into contact with the charger 30 when the secondary battery 4 of the capsule medication device 10 is charged is on the opposite side of the metal cover 22. That is, as illustrated in FIG. 4, the storage sheet 20 is placed on the placement surface 30a of the charger 30 so as the portion of the storage sheet 20 opposite to the metal cover 22 to face the placement surface 30a of the charger 30. When charging is performed in this state, it is less likely to receive an effect by an electromagnetic wave coming from the side on which the metal cover 22 is provided, that is, the side opposite to the charger 30 across the storage sheet 20, which enables efficient charging.

In the capsule medication device charging system 100 of the present disclosure, the charging of the capsule medication device 10 stored in the storage 21 of the storage sheet 20 can be performed by a patient himself or herself who takes a medicine or at a pharmacy at which the capsule medication device 10 is handed with a medicine to a patient.

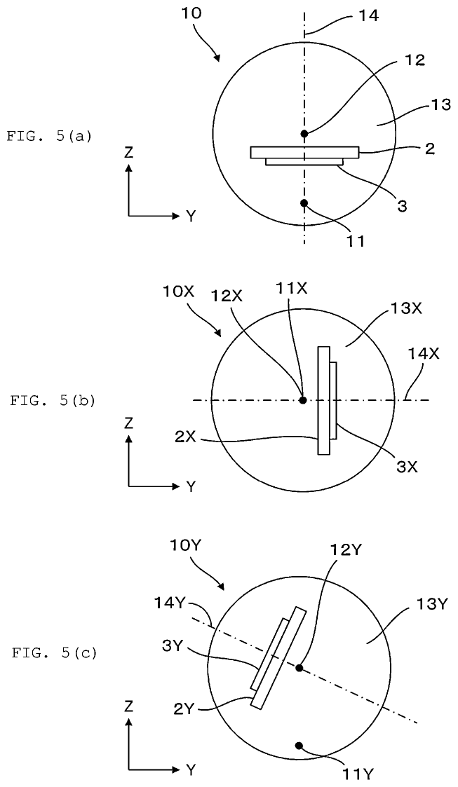

As described above, in the capsule medication device 10 according to the present exemplary embodiment, the gravity center 11 is not located at the center 12 of the cut face 13 on a plane that passes through the gravity center 11 and is orthogonal to the longitudinal direction of the capsule medication device 10. Moreover, the gravity center 11 overlaps the substrate 2 in a view along a direction orthogonal to the substrate 2. With such a configuration, when the capsule medication device 10 is placed on the placement surface 30a of the charger 30, wireless power supply can be efficiently performed, such that the secondary battery 4 is efficiently charged. This will be described with reference to FIG. 5.

FIG. 5(a) is a side sectional view schematically illustrating an orientation of the capsule medication device 10 according to the present exemplary embodiment, in which orientation the capsule medication device 10 is charged. FIG. 5(b) is a side sectional view schematically illustrating an orientation of a capsule medication device 10X in which a gravity center 11X is on a center 12X of a cut face 13X, in which orientation the capsule medication device 10X is charged. FIG. 5(c) is a side sectional view schematically illustrating an orientation of a capsule medication device 10Y in which the gravity center 11Y is not located at a center 12Y of a cut face 13Y described above and is not located to overlap a substrate 2Y in a view along a direction orthogonal to the substrate 2Y, in which orientation the capsule medication device 10Y is charged. FIGS. 5(a) to 5(c) each illustrate a state, although the charger 30 is omitted, in which the capsule medication devices 10, 10X, and 10Y are each placed on the placement surface 30a of the charger 30 as in FIG. 4. That is, in FIGS. 5(a) to 5(c), the charger 30 is located vertically below the capsule medication devices 10, 10X, and 10Y.

The capsule medication device 10X illustrated in FIG. 5(b) and the capsule medication device 10Y illustrated in FIG. 5(c) are not the capsule medication device of the present disclosure. Among the components of the capsule medication device 10X, a component corresponding to one of the components of the capsule medication device 10 will be described with the reference symbol of the corresponding component with X added. Among the components of the capsule medication device 10Y, components corresponding to one of the components of the capsule medication device 10 will be described with the reference symbol of the corresponding component with Y added.

As illustrated in FIG. 5(b), when the gravity center 11X of the capsule medication device 10X is on the center 12X of the cut face 13X described above, the capsule medication device 10X placed on the placement surface 30a of the charger 30 is rotatable about a center line that passes through the gravity center 11X and is parallel to the longitudinal direction. Therefore, the capsule medication device 10X easily rotates in the storage 21 of the storage sheet 20, and the relative positional relationship between the power transmission coil 31 of the charger 30 and the power receiving coil 3X of the capsule medication device 10X is not uniquely determined. Therefore, as illustrated in FIG. 5(b), the substrate 2X of the capsule medication device 10X may have an orthogonal positional relationship with the placement surface 30a of the charger 30. In this case, the power receiving coil 3X provided on the substrate 2X and the power transmission coil 31 of the charger 30 have an orthogonal positional relationship that reduces power supply efficiency of wireless power supply, and thus the charging efficiency of a secondary battery 4X is also reduced. In addition, as described above, since the capsule medication device 10X easily rotates in the storage 21 of the storage sheet 20, the change of orientation causes a variation in charging of the secondary batteries 4X of a plurality of capsule medication devices 10X.

In addition, as illustrated in FIG. 5(c), when the gravity center 11Y of the capsule medication device 10Y is not located at the center 12Y of the cut face 13Y described above and does not overlap the substrate 2Y in a view along a direction orthogonal to the substrate 2Y, the angle between the placement surface 30a of the charger 30 and the substrate 2Y is large, and thus the amount of magnetic flux passing through the power receiving coil 3Y among the magnetic flux generated by the current flowing in the power transmission coil 31 is small. Therefore, the power supply efficiency of wireless power supply decreases and leads to a low efficiency of charging the secondary battery 4X.

In contrast, as for the capsule medication device 10 of the present exemplary embodiment in which the gravity center 11 is not located at the center 12 of the cut face 13 described above and also overlaps the substrate 2 in a view along a direction orthogonal to the substrate 2, the relative positional relationship between the power transmission coil 31 and the power receiving coil 3 can be uniquely determined by placing the capsule medication device 10 on the charger 30. That is, since the gravity center 11 is located vertically below the center 12 of the cut face 13 described above, the angle between the placement surface 30a of the charger 30 and the substrate 2 does not change. Therefore, variation in wireless charging of the secondary batteries 4 of a plurality of capsule medication devices 10 stored in a plurality of storages 21 of the storage sheet 20 is suppressed.

In addition, the capsule medication device 10 of the present exemplary embodiment, when placed on the charger 30, does not have the positional relationship as illustrated in FIG. 5(b), that is, the positional relationship in which the power receiving coil 3 is orthogonal to the power transmission coil 31, so that wireless power supply can be efficiently performed, and thus the secondary battery 4 is also efficiently charged.

In addition, as illustrated in FIG. 5(a), the gravity center 11 of the capsule medication device 10 is preferably located on a reference line 14 passing through the center 12 of the cut face 13 described above and extending in a direction orthogonal to the substrate 2. When the gravity center 11 of the capsule medication device 10 is located on the reference line 14 passing through the center 12 of the cut face 13 described above and extending in the direction orthogonal to the substrate 2, the placement surface 30a of the charger 30 and the substrate 2 of the capsule medication device 10, more specifically, a main surface of the substrate 2 on which the power receiving coil 3 is provided become parallel when the capsule medication device 10 is placed on the placement surface 30a of the charger 30. With the power receiving coil 3 and the power transmission coil 31 having a parallel positional relationship, wireless power supply is performed with its highest power supply efficiency, and thus the secondary battery 4 is also charged most efficiently.

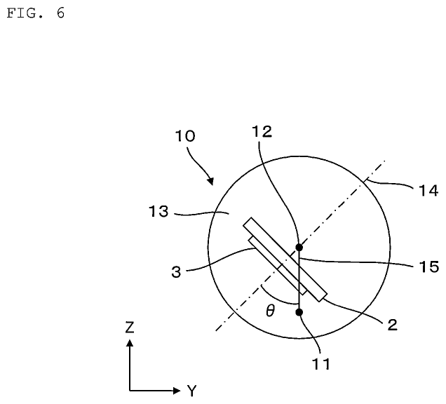

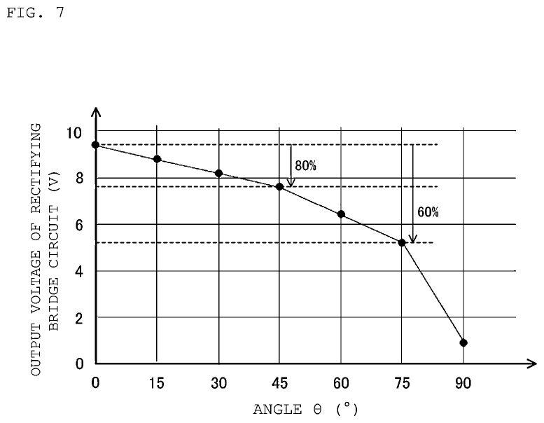

The location of the gravity center 11 was moved to change the angle θ (see FIG. 6) between a line 15 connecting the gravity center 11 of the capsule medication device 10 and the center 12 of the cut face 13 described above and the reference line 14 passing through the center 12 of the cut face 13 and extending in a direction orthogonal to the substrate 2, and a voltage was examined while wireless power supply is performed using the charger 30. The examined results are shown in FIG. 7. In FIG. 7, the horizontal axis represents the angle θ described above, and the vertical axis represents the output voltage of the rectifying bridge circuit disposed subsequent to the power receiving coil 3. For a high output voltage of the rectifying bridge circuit, a booster circuit for fully charging the secondary battery 4 is not needed, and the number of parts can be reduced. The secondary battery 4 is charged from the rectifying bridge circuit desirably with a linear regulator (LDO) for stably charging the secondary battery 4, a charge monitoring IC, or the like provided. Also, with a linear regulator provided, an effect resulting from a high output voltage of the rectifying bridge circuit is obtained.

As shown in FIG. 7, the output voltage of the rectifying bridge circuit becomes its highest when the angle θ between the line 15 connecting the gravity center 11 of the capsule medication device 10 and the center 12 of the cut face 13 described above and the reference line 14 is 0°, that is, when the gravity center 11 of the capsule medication device 10 is located on the reference line 14 as illustrated in FIG. 5(a). Meanwhile, the output voltage of the rectifying bridge circuit becomes its lowest when the angle θ is 90°, that is, when the substrate 2 and the placement surface 30a of the charger 30 have an orthogonal positional relationship as illustrated in FIG. 5(b).

As illustrated in FIG. 7, when the angle θ is 75° or less, the output voltage of the rectifying bridge circuit is 60% or more of the output voltage of the rectifying bridge circuit with the angle θ of 0°. Therefore, the angle θ is preferably between the line 15 connecting the gravity center 11 of the capsule medication device 10 and the center 12 of the cut face 13 described above and the reference line 14 passing through the center 12 of the cut face 13 and extending in the direction orthogonal to the substrate 2 is 75° or less.

As illustrated in FIG. 7, the output voltage of the rectifying bridge circuit with the angle θ of 45° or less is 80% or more of the output voltage of the rectifying bridge circuit with the angle θ of 0°. Therefore, the angle θ is preferably between the line 15 connecting the gravity center 11 of the capsule medication device 10 and the center 12 of the cut face 13 described above and the reference line 14 passing through the center 12 of the cut face 13 and extending in the direction orthogonal to the substrate 2 is 45° or less.

Also, for a large angle θ, the output voltage of the rectifying bridge circuit can be increased by increasing the voltage applied to the power transmission coil 31 of the charger 30. However, increasing the voltage applied to the power transmission coil 31 intensifies an electromagnetic field and may affect other electronic components. Therefore, to increase the output voltage of the rectifying bridge circuit without increasing the voltage applied to the power transmission coil 31, the angle e is preferably set to 75° or less, more preferably, 45° or less.

In general, it should be noted that the present disclosure is not limited to the above exemplary embodiment, and various applications and modifications can be made within the scope of the present invention.

A capsule medication device, and a capsule medication device charging system according to the present disclosure are as follows.

<1> A capsule medication device is provided that includes a housing having a capsule shape; a substrate disposed inside the housing; a power receiving coil on the substrate; and a secondary battery configured to be charged with power received by the power receiving coil. In this aspect, a gravity center of the capsule medication device is not located at a center of a cut face when the capsule medication device is cut along a plane that passes through the gravity center and is orthogonal to a longitudinal direction of the capsule medication device. Moreover, the gravity center overlaps the substrate in a direction orthogonal to the substrate.

<2> The capsule medication device according to <1>, in which an angle between a line connecting the gravity center of the capsule medication device and the center of the cut face and a reference line passing through the center of the cut face and extending in a direction orthogonal to the substrate is 75° or less.

<3> The capsule medication device according to <2>, in which the angle between the line connecting the gravity center of the capsule medication device and the center of the cut face and the reference line is 45° or less.

<4> The capsule medication device according to any one of <1> to <3>, in which the gravity center of the capsule medication device is located on a reference line passing through the center of the cut face and extending in the direction orthogonal to the substrate.

<5> The capsule medication device according to any one of <1> to <4>, further including a biological information acquisition unit configured to acquire biological information; and a signal transmission unit configured to transmit the biological information acquired by the biological information acquisition unit.

<6> The capsule medication device according to <5> in which the signal transmission unit is a piezoelectric element.

<7> The capsule medication device according to any one of <1> to <6>, wherein the housing is an assembly of a first split housing and a second split housing, and a weight of the first split housing and a weight of the second split housing are different.

<8> A capsule medication device charging system including a storage sheet including a plurality of storages and a metal cover that covers openings of the plurality of storages; the capsule medication device according to any one of <1> to <7> that is configured to be stored in one of the plurality of storages of the storage sheet; and a charger that includes a power transmission coil, the storage sheet that stores the capsule medication device in the one of the plurality of storages being placed on the charger.

<9> The capsule medication device charging system according to <8>, in which a portion of the storage sheet that is brought into contact with the charger when the secondary battery is charged is on a side opposite to the metal cover.

### DESCRIPTION OF REFERENCE SYMBOLS

1: Housing
1a: First split housing
1b: Second split housing
2: Substrate
3: Power receiving coil
4: Secondary battery
5: Biological information acquisition unit
6: Signal transmission unit
10: Capsule medication device
11: Gravity center of capsule medication device
12: Center of a cut face on a plane that cuts a capsule medication device, passes through a gravity center, and is orthogonal to a longitudinal direction
13: Cut face on a plane that cuts a capsule medication device, passes through a gravity center, and is orthogonal to a longitudinal direction
14: Reference line
20: Storage sheet
21: Storage
22: Metal cover
30: Charger
31: Power transmission coil
100: Capsule medication device charging system

1. A capsule medication device comprising:
a housing having a capsule shape;
a substrate disposed inside the housing;
a power receiving coil on the substrate; and
a secondary battery configured to be charged with power received by the power receiving coil,
wherein the capsule medication device has a gravity center that is not located at a center of a cut face when the capsule medication device is cut along a plane that passes through the gravity center and is orthogonal to a longitudinal direction of the capsule medication device, and
wherein the gravity center of the capsule medication device overlaps the substrate in a direction orthogonal to the substrate.

2. The capsule medication device according to claim 1, wherein an angle between a line connecting the gravity center of the capsule medication device and the center of the cut face and a reference line passing through the center of the cut face and extending in a direction orthogonal to the substrate is 75° or less.

3. The capsule medication device according to claim 2, wherein the angle between the line connecting the gravity center of the capsule medication device and the center of the cut face and the reference line is 45° or less.

4. The capsule medication device according to claim 1, wherein the gravity center of the capsule medication device is on a reference line passing through the center of the cut face and extending in the direction orthogonal to the substrate.

5. The capsule medication device according to claim 1, further comprising:
a biological information acquisition unit configured to acquire biological information; and
a signal transmission unit configured to transmit the biological information acquired by the biological information acquisition unit.

6. The capsule medication device according to claim 5, wherein the signal transmission unit is a piezoelectric element.

7. The capsule medication device according to claim 1, wherein the housing is an assembly that includes a first split housing and a second split housing.

8. The capsule medication device according to claim 7, wherein a weight of the first split housing is different than a weight of the second split housing.

9. The capsule medication device according to claim 8, wherein a weight is provided in one of the first split housing and the second split housing to configure the different weights of the first and second split housings.

10. The capsule medication device according to claim 9, wherein at least a portion of the weight overlaps the substrate in the direction orthogonal to the substrate.

11. The capsule medication device according to claim 9, wherein an entirety of the weight overlaps the substrate in the direction orthogonal to the substrate.

12. The capsule medication device according to claim 9, wherein the substrate is provided in one of the first split housing and the second split housing.

13. A capsule medication device charging system comprising:
a storage sheet that includes a plurality of storages and a metal cover that covers openings of the plurality of storages;
the capsule medication device according to claim 1 that is configured to be stored in one of the plurality of storages of the storage sheet; and
a charger that includes a power transmission coil,
wherein the storage sheet that configured to store the capsule medication device in the one of the plurality of storages is configured to be placed on the charger.

14. The capsule medication device charging system according to claim 13, wherein a portion of the storage sheet that is brought into contact with the charger when the secondary battery is charged is on a side opposite to the metal cover.

15. A capsule medication device comprising:
a housing having an elongated shape extending in a longitudinal direction;
a substrate inside the housing;
a power receiving coil on the substrate; and
a secondary battery,
wherein the capsule medication device has a gravity center that is not located at a center of a circular cross-sectional plane that is orthogonal to the longitudinal direction of the elongated shape, and
wherein the gravity center of the capsule medication device overlaps the substrate in a direction orthogonal to the substrate.

16. The capsule medication device according to claim 15, wherein the elongated shape of the housing is a capsule shape.

17. The capsule medication device according to claim 15, wherein an angle between a line connecting the gravity center of the capsule medication device and the center of the circular cross-sectional plane and a reference line passing through the center of the circular cross-sectional plane and extending in a direction orthogonal to the substrate is 75° or less.

18. The capsule medication device according to claim 17, wherein the angle between the line connecting the gravity center of the capsule medication device and the center of the circular cross-sectional plane and the reference line is 45° or less.

19. The capsule medication device according to claim 15, wherein the gravity center of the capsule medication device is on a reference line passing through the center of the circular cross-sectional plane and extending in the direction orthogonal to the substrate.

20. The capsule medication device according to claim 15, further comprising:
a biological information acquisition unit configured to acquire biological information; and
a signal transmission unit configured to transmit the biological information acquired by the biological information acquisition unit.
